(19) United States
(12) Patent Application Publication
(10) Pub. No.: US 2020/0268938 A1
(43) Pub. Date: Aug. 27, 2020

(54) NOVEL BONE PUTTY COMPOSITIONS AND METHODS OF USE THEREOF

(71) Applicant: HS PHARMACEUTICALS, LLC, Greenville, SC (US)

(72) Inventor: Daniel NICKELS, Greenville, SC (US)

(21) Appl. No.: 16/089,613

(22) PCT Filed: Mar. 31, 2017
(86) PCT No.: PCT/US2017/025453

§ 371 Date: Sep. 28, 2018

Related U.S. Application Data
(60) Provisional application No. 62/317,217, filed on Apr. 1, 2016.

(51) Int. Cl. A61L 27/36; A61L 27/12; A61L 27/54; A61L 27/50

(52) U.S. Cl. CPC A61L 27/3608; A61L 27/12; A61L 27/54; A61L 2300/102; A61L 2430/02; A61L 2300/414; A61L 2300/232; A61L 27/505

(57) ABSTRACT

The present disclosure relates to novel bone putty compositions comprising a silicate-containing preparation and bone and/or a bone surrogate material, and methods for making and using the same in healing damaged bone in a subject in need thereof. The present compositions may also promote healing of soft tissue contiguous with the treated damaged bone which is in contact with the present compositions. In some embodiments, the present compositions are stabilized, and contain silicate-containing particles having a mean diameter of from about 1 to about 20 nm. Such particles may import unique healing characteristics to the composition, both with respect to the damaged bone and the surrounding soft tissue. The present compositions and methods may find particular utility in the oral cavity of the subject.

### CROSS REFERENCE TO RELATED APPLICATIONS

The present application claims the benefit of priority to U.S. Provisional Application No. 62/317,217, filed on Apr. 1, 2016, the contents of which are hereby incorporated by reference in their entirety.

### FIELD

The present disclosure provides novel bone putty compositions and methods for using the same in the repair and/or replacement of damaged of lost bone in a subject in need thereof.

### BACKGROUND

The present disclosure relates to bone putty employed to repair or replace damaged bone. Materials, such as bone putty, for bone repair and bone void filling should ideally be formable so that it can fill and conform to irregular shaped and sized bone defects. However, once implanted it should set hard so that the implant material maintains its shape and, when needed, can bear loads. The material should not break up and needs to be tough. Furthermore, in certain circumstances, the material should allow rapid bone in-growth, be degradable and have the potential to be fully replaced by native growth bone.

In the oral cavity, where bone loss due to periodontal disease is common, the oral cavity is particularly laden with bacteria. In order to facilitate bone repair the material may incorporate a drug or bioactive molecule which is released to stimulate bone healing and repair. A number of bone putty materials are known, but very few meet all the ideal requirements. Poly(methyl methacrylate) bone cements are widely used to fixate joint replacements but these materials are non-porous and non-degradable so they are not replaced by bone. In addition, when the cement cures, heat is generated and the temperature of the material can rise to 90° C. or above. This can damage any drug material or bioactive agent which may have been added to the cement, particularly if the bioactive agent consists of proteins such as bone morphogeneic protein (BMP) etc.

Calcium phosphate ceramics, such as hydroxyapatite and tricalcium phosphate, are widely used for bone putties. These fillers are available in a number of forms. For example, the use of dense and porous granules is known. These can be used to fill irregular shaped defects and allow bone growth into and between the granules. However, they cannot maintain a specific shape or form, and tend to migrate if not fully contained.

Porous blocks in pre-formed shapes are also known. However, whilst these kinds of fillers maintain their shape, they cannot be used to fill irregular sized/shaped defects. In addition, it is not easy to incorporate a drug or bioactive material into these ceramics as high temperatures are required in their manufacture. Drugs or bioactive agents can be adsorbed or coated onto the surface of these ceramics but they tend to be released very quickly.

Calcium phosphate cements have also been used as bone putty. These kinds of fillers have the advantage of being mouldable, and even injectable, and once in place they set hard. However, whilst they may contain micropores, these tend not to allow significant levels of bone ingrowth. Some calcium phosphate cements have macropores but these generally compromise the mechanical strength of the material. In addition, calcium phosphate ceramics (blocks, cements, etc.) generally tend to form brittle materials.

There have been attempts to produce bone putties which harden in-situ; these combine ceramic granules with a polymer. For example, US 2010/0041770 (incorporated herein by reference in its entirety) discloses a composite material formed by mixing a polymer phase with a solvent, adding a bioresorbable ceramic phase, and thereafter allowing the solvent to diffuse out of the polymer in the presence of water, to cause solidification of the polymer phase. The composite formed does not have initial porosity for rapid bone in-growth, though pores may form later by degradation of one of the phases.

US 2005/0251266 (incorporated herein by reference in its entirety) discloses a moldable composite comprising ceramic granules coated with a biocompatible polymer and a plasticizer such that the polymer is initially deformable and then hardens upon removal of the plasticizer by placing in water. However, coating the granules is difficult and the specialist processes which need to be employed leads to an increase in cost. In addition, since all the granules are coated with polymer there is a delay in the osteoconductive effect of the bioceramic granules until at least some of the polymer degrades.

Thus, there exists a need for biocompatible bone putties with rapid healing characteristics that promote healing of adjacent tissues.

### SUMMARY OF THE DISCLOSURE

The present disclosure seeks to address at least some of these problems by providing a bone putty for repairing or replacing damaged bone. The present compositions are moldable/formable; set to a hard and tough material; are able to bear loads; in some embodiments allow for rapid bone in-growth; and where bone ingrowth is desired, are biodegradable and substantially replaced by bone without substantially compromising the structural integrity of the site of application. The present compositions and methods are particularly suited to use in the oral cavity, as the present compositions exhibit unexpected anti-bacterial properties.

In one aspect, the present disclosure provides compositions and methods for providing a dental implant in a subject in need thereof. For dental implants to be successful, the jawbone must have enough bone to support them. Tooth loss often leads to more loss of bone over time. The tooth loss may be caused by: (i) periodontal (gum) disease: (ii) dental caries (cavities) and infection: (iii) injury or trauma: or (iv) a defect in development. If the bone under the patient's gum is not tall enough, not wide enough or both, the patient needs a procedure to add bone to the jaw before dental implants can be placed. A variety of procedures are used to build bone so that dental implants can be placed. These procedures typically involve grafting (adding) bone or bonelike materials to the jaw. The graft can be the patient's own bone or processed bone (off the shelf) obtained from a cadaver. After grafting, the patient has to wait several months for the grafted material to fuse with the existing bone. The compositions and methods of the present invention make the bone graft less susceptible to falling out (or alternatively stated, the present compositions and methods improve retention of the bone graft in the jaw), provide rapid bone growth at the site of the bone graft, and/or reduce or prevent infections at the site of the bone graft.

In a first aspect, the present disclosure provides a composition for repairing or replacing bone in a subject in need thereof, the composition comprising a silicate-containing preparation, and bone and/or a bone surrogate material. In one embodiment, the composition further comprises a stabilizing agent which retards the growth of silicate gels and polysilicates from the silicate-containing particles. The stabilizing agent can be incorporated into the compositions of the present invention either before, during or after combining the silicate-containing preparation and the bone and/or bone surrogate material to form the composition. If provided before, the stabilizer can be added to the silicate-containing preparation, or to the bone and/or bone surrogate material. If provided during, the stabilizer can be added to the silicate-containing preparation and the bone and/or bone surrogate material as they are mixed. If after, the stabilizer can be added to combination of silicate-containing preparation and the bone and/or bone surrogate material after they are mixed.

In another aspect, the present disclosure provides a method for repairing or replacing damaged bone in a subject in need thereof, the method comprising applying a composition as described herein to damaged or missing bone in the subject.

In yet another aspect, the present disclosure provides a method for bone regeneration or bone augmentation in a subject in need thereof, the method comprising applying a bone putty composition comprising a silicate-containing preparation and a bone and/or bone surrogate material to the site of damaged bone.

In another aspect, the present disclosure provides a method for preparing a composition for repairing or replacing bone, comprising combining a silicate-containing preparation and a bone and/or a bone surrogate material. In yet another embodiment, the present disclosure provides a method for preparing a composition for repairing or replacing bone, comprising combining the silicate-containing preparation, a stabilizing agent and a bone and/or a bone surrogate material.

In another embodiment, the present disclosure provides composition for repairing or replacing bone in a subject in need thereof made by the methods described herein.

The bone putty compositions of the present disclosure comprising a silicate-containing preparation, and bone and/or a bone surrogate material, when applied to the site of damaged or missing bone, provide support for the grafted bone material so that the grafted bone material stays in place for a substantially long period of time and provide rapid bone regeneration. Additionally, the compositions of the present disclosure render the grafted bone material less prone to be infected.

### DETAILED DESCRIPTION OF THE DISCLOSURE

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity: for example, “a kinase inhibitor” refers to one or more kinase inhibitors or at least one kinase inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “treat,” “treated,” “treating” or “treatment” includes the diminishment or alleviation of at least one symptom associated or caused by the state, disorder or disease being treated. Treatment can be diminishment of one or several symptoms of a disorder or complete eradication of a disorder or a disease.

As used herein, the term “bone surrogate” refers to any material which simulates the characteristics of natural bone in situ, including synthetic hydroxyapatite and Ca- and/or Si-containing sol gel systems.

As used herein, the term “subject,” “individual” or “patient” is used interchangeably and refers to a vertebrate, preferably a mammal. Non-limiting examples include mice, dogs, rabbits, farm animals, sport animals, pets, and humans.

As used herein, “therapeutically effective amount” or an “effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the present invention. All references cited herein are incorporated by reference in their entirety.

### Bone Putty Compositions

In a first aspect, the present disclosure provides a composition for repairing, replacing, regenerating, or augmenting bone in a subject in need thereof, the composition comprising a silicate-containing preparation, and bone and/or a bone surrogate material. Suitable silicate-containing compositions include those disclosed in US Pat. Appl. Pub. No. 2009/0130230 and WO 2015/121666, each of which is incorporated herein by reference in its entirety.

In certain embodiments, the silicate-containing preparation comprises silicate-containing particles. Such silicate particles can have a particle size distribution such that the mean diameter of the particles is from about 1 nm to about 1000 nm, 1 nm to about 100 nm, 1 nm to about 90 nm, 1 nm to about 80 nm, 1 nm to about 70 nm, 1 nm to about 60 nm, 1 nm to about 50 nm, 1 nm to about 40 nm, 1 nm to about 30 nm, 1 nm to about 20 nm, or any other value or range of values therein. In some embodiments, the silicate-containing particles have a mean diameter of from about 0.1 nm to about 100 nm.

In some embodiments, the silicate-containing preparation may constitute from about 1 wt % to about 90 wt % of the composition. In other embodiments, the silicate-containing preparation may constitute from about 1 wt % to about 2 wt %, about 2 wt % to about 3 wt %, about 3 wt % to about 4 wt %, about 4 wt % to about 5 wt %, about 5 wt % to about 6 wt %, about 6 wt % to about 7 wt %, about 7 wt % to about 8 wt %, about 8 wt % to about 9 wt %, about 9 wt % to about 10 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 45 wt % to about 50 wt %, about 50 wt % to about 60 wt %, about 60 wt % to about 70 wt %, about 70 wt % to about 80 wt %, about 80 wt % to about 90 wt %, or any other value or range of values therein.

In some embodiments, the present compositions may also comprise a stabilizing agent. Without being bound by any particular theory, it is believed that the stabilizing agent retards the growth of silicate gels and polysilicates from the silicate-containing particles. Such stabilizers and compositions comprising the same are described in WO 2015/121666, and are suitable for use in the present compositions and methods. In some embodiments, the stabilizing agent is sucrose or polyethylene glycol (PEG). In certain embodiments, the stabilizing agent is PEG.

In some embodiments, the stabilizer may be from about 1 wt % to about 50 wt % of the composition. In other embodiments, the stabilizer may be from about 1 wt % to about 2 wt %, about 2 wt % to about 3 wt %, about 3 wt % to about 4 wt %, about 4 wt % to about 5 wt %, about 5 wt % to about 6 wt %, about 6 wt % to about 7 wt %, about 7 wt % to about 8 wt %, about 8 wt % to about 9 wt %, about 9 wt % to about 10 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 45 wt % to about 50 wt %, or any other value or range of values therein.

In some embodiments, the bone in the present compositions may be obtained from the subject. For example, in oral surgical procedures, bone may be taken from a remote part of the jaw, removed and macerated to be used in the present compositions. Alternatively, the bone may be obtained from a donor (e.g., a living donor). In another embodiment, the bone is obtained from a cadaver. In some embodiments, the bone comprises hydroxyapatite. In other embodiments, the bone surrogate material comprises hydroxyapatite, for example any conventional hydroxyapatite containing materials used in bone repair or reconstruction. In certain embodiments (e.g., as described in U.S. Pat. No. 8,303,971, incorporated herein by reference in its entirety), the bone surrogate material (e.g., hydroxyapatite) is formed in situ via a sol gel process.

In some embodiments, the bone and/or bone surrogate material may be from about 1 wt % to about 90 wt % of the composition. In other embodiments, the bone and/or bone surrogate material may be from about 1 wt % to about 2 wt %, about 2 wt % to about 3 wt, about 3 wt % to about 4 wt %, about 4 wt % to about 5 wt %, about 5 wt % to about 6 wt %, about 6 wt % to about 7 wt %, about 7 wt % to about 8 wt %, about 8 wt % to about 9 wt %, about 9 wt % to about 10 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 45 wt % to about 50 wt %, about 50 wt % to about 60 wt %, about 60 wt % to about 70 wt %, about 70 wt % to about 80 wt %, about 80 wt % to about 90 wt %, or any other value or range of values therein.

In some embodiments, the present compositions may include a pharmaceutically acceptable carrier to assist in application of the composition. Thus, in some embodiments, the composition can a gel or an ointment component, for example as part of the silicate-containing preparation.

In one embodiment, the present disclosure provides a pharmaceutical composition and/or pharmaceutical combination comprising a silicate-containing preparation, and bone and/or a bone surrogate material, and optionally a stabilizer.

In one embodiment, any pharmaceutical compositions and/or pharmaceutical combinations as disclosed herein comprising a silicate-containing preparation, and bone and/or a bone surrogate material, and optionally a stabilizer further comprises a pharmaceutically acceptable excipient. The pharmaceutically acceptable excipients are added to the formulation for a variety of purposes.

The compositions disclosed herein can be formulated in accordance with the routine procedures adapted for the desired delivery of bone repair or reconstruction compositions. Accordingly, the compositions disclosed herein can take such forms as suspensions, gels, pastes, in oily or aqueous vehicles, and can contain formulatory agents such as suspending, stabilizing and/or dispersing agents. The compositions disclosed herein can also be formulated as a preparation for implantation. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient (e.g., the silicate nanoparticles) can be in powder form or in a concentrated solution form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

In one embodiment, the compositions of the present invention can include a pharmaceutically acceptable carrier. Pharmaceutically acceptable carriers are well known to those skilled in the art and include, but are not limited to, water, or buffered and/or isotonic aqueous compositions, for example about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

Solid and liquid compositions and/or combinations may also be dyed using any pharmaceutically acceptable colorant to improve their appearance and/or facilitate patient identification of the product and unit dosage level.

In liquid pharmaceutical compositions and/or combinations may be prepared using compositions as described herein and any other solid excipients where the components are dissolved or suspended in a liquid carrier such as water, vegetable oil, alcohol, polyethylene glycol, propylene glycol, or glycerin.

### Methods for Repairing Damaged Bone

In another embodiment, the present disclosure provides a method for repairing or replacing damaged bone in a subject in need thereof, the method comprising applying a composition as described herein to damaged or missing bone in the subject. In some embodiments, it may prove useful to remove at least a portion of the damaged bone prior to applying the compositions described herein. Such removal of bone may promote bonding and/or adhesion of the composition to the damaged bone.

In another embodiment, the present disclosure provides a method for providing a dental implant in a subject in need thereof, the method comprising applying a bone putty composition comprising a silicate-containing preparation and a bone or bone surrogate material to the site of dental implant.

In one embodiment, the present methods may also promote healing of soft tissue underlying or contiguous with the composition. Thus, during bone repair, surrounding tissues may also experience rapid healing. Typically, an area of bone needing repair is exposed via incision, and a composition as described herein is applied to the damaged area. Alternatively, a composition may be applied via syringe to the surface of the damaged area. In some embodiments, the repaired area can be covered by a patch or film, comprising either natural, naturally derived, or synthetic materials to facilitate healing. Such patches or films can themselves be treated with the silicate-containing materials described herein to facilitate healing, either of the bone, the surrounding soft tissue, or both. In a particular embodiment, the patch or film is a collagen film, for example a collagen film coated or contacted (e.g. dipped) in any of the silicate-containing formulations as described herein.

The method of administration of the present compositions can be any method commonly known in the art of medicine or dentistry.

The compositions and methods of the present invention provide faster bone growth or bone regeneration when applied to the damaged bone. For example, in certain embodiments, bone growth in subjects treated with the compositions and methods of the present invention is about 7 days, 10 days, 15 days, 20 days, 25 days, 4 weeks, 6 weeks, 1 month, 2 months, 3 months, 4 months, 5 months, or 6 months, including values and ranges therebetween, faster than the untreated subjects or subjects treated with a reference composition.

In one embodiment, the reference composition is a composition that does not comprise a silicate-containing preparation.

The compositions and methods of the present invention provide a faster healing of the site of damaged bone. For example, in certain embodiments, in subjects treated with the compositions and methods of the present invention, the site of damaged bone heals about 7 days, 8 days, 9 days, 10 days, 12 days, 15 days, 20 days, 25 days, 4 weeks, 6 weeks, 1 month, 2 months, 3 months, 4 months, 5 months, or 6 months, including values and ranges therebetween, faster than the untreated subjects or subjects treated with a reference composition. For example, in one embodiment, in subjects treated with the compositions and methods of the present invention, the site of damaged bone heals in about 9 days compared to 14 days in subjects treated with compositions lacking a silicate-containing preparation.

The compositions and methods of the present invention keep the bone graft in place for a longer period of time compared to untreated subjects or subjects treated with a reference composition. For example, in certain embodiments, in subjects treated with the compositions and methods of the present invention, bone graft stays in place for at least about 7 days more, 10 days more, 15 days more, 20 days more, 25 days more, 4 weeks more, 6 weeks more, 1 month more, 2 months more, 3 months more, 4 months more, 5 months more, or 6 months, including values and ranges therebetween, more compared to untreated subjects or subjects treated with a reference composition.

The compositions and methods of the present invention prevent or reduce the chances of infections at the site of bone graft and/or in the bone graft itself.

In some embodiments, the bone regenerated using the compositions and methods of the present invention show superior bone mineral content (BMC) and bone density. For example, in certain embodiments, the BMC of the subjects treated using the compositions and methods of the present invention is about 2%, 3%, 4%, 5%, 10%, 15%, 20%, 25%, 30%, 35%, 40%, 50%, 55%, 60%, 65%, 70%, 75%, 80%, 85%, 90%, or 95%, including values and ranges therebetween, more compared to untreated subjects or subjects treated with a reference composition.

In some embodiments, the bone density of the subjects treated using the compositions and methods of the present invention is about 2%, 3%, 4%, 5%, 10%, 15%, 20%, 25%, 30%, 35%, 40%, 50%, 55%, 60%, 65%, 70%, 75%, 80%, 85%, 90%, or 95%, including values and ranges therebetween, more compared to untreated subjects or subjects treated with a reference composition.

### Methods for Preparing Bone Putty Compositions

1. Methods for preparing the present compositions are those as-described in US Pat. Appl. Pub. No. 2009/0130230 and WO 2015/121666. A method for preparing a composition for repairing or replacing bone, comprising combining a silicate-containing preparation and a bone and/or a bone surrogate material. In some embodiments, the present disclosure provides a method for preparing a composition for repairing or replacing bone, comprising combining the silicate-containing preparation, an optional stabilizing agent and a bone and/or a bone surrogate material. In some embodiments, the composition further comprises a stabilizer. In some embodiments, the stabilizer is added in an amount effective to provide silicate-containing particles having a mean diameter of from about 1 nm to about 20 nm.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. A composition for repairing or replacing bone in a subject in need thereof, the composition comprising:
a silicate-containing preparation, and
bone and/or a bone surrogate material.

2. The composition of claim 1, wherein the silicate-containing preparation comprises silicate-containing particles.

3. The composition of claim 2, wherein the silicate-containing particles have a mean diameter of from about 0.1 nm to about 100 nm.

4. The composition of claim 3, wherein the silicate-containing particles have a mean diameter of from about 1 nm to about 50 nm.

5. The composition of claim 4, wherein the silicate-containing particles have a mean diameter of from about 1 nm to about 20 nm.

6. The composition of claim 2, further comprising a stabilizing agent.

7. The composition of claim 3, wherein the stabilizing agent retards the growth of silicate gels and polysilicates from the silicate-containing particles.

8. The composition of claim 1, where the stabilizing agent is sucrose or polyethylene glycol (PEG).

9. The composition of claim 1, wherein the bone is obtained from the subject.

10. The composition of claim 1, wherein the bone is obtained from a donor.

11. The composition of claim 1, wherein the bone is obtained from a cadaver.

12. The composition of claim 1, wherein the bone comprises hydroxyapatite.

13. The composition of claim 1, wherein the bone surrogate material comprises hydroxyapatite.

14. The composition of claim 13, wherein the hydroxyapatite is formed in situ via a sol gel process.

15. The composition of claim 1, wherein the composition comprises a pharmaceutically acceptable carrier.

16. The composition of claim 1, wherein the composition is a gel or an ointment.

17. A method for repairing or replacing damaged bone in a subject in need thereof, the method comprising applying the composition of claim 1 to damaged or missing bone in the subject.

18. The method of claim 17, further comprising prior to the applying, removing at least a portion of the damaged bone.

19. The method of claim 17, wherein the removing promotes bonding and/or adhesion of the composition to the damaged bone.

20. The method of claim 17, further comprising promoting healing of soft tissue underlying or contiguous with the composition.

21. The method of claim 17, comprising exposing the damaged bone and applying the composition thereto.

22. The method of claim 21, wherein the composition is applied via syringe to the surface of the damaged bone.

23. The method of claim 21, wherein the composition is applied via syringe to one or more fissures in the damaged bone.

24. A method for preparing a composition for repairing or replacing bone, comprising combining a silicate-containing preparation and a bone and/or a bone surrogate material.

25. A method for preparing a composition for repairing or replacing bone, comprising combining the silicate-containing preparation, a stabilizing agent and a bone and/or a bone surrogate material.

26. The method of claim 25, wherein the stabilizing composition is added in an amount effective to provide silicate-containing particles having a mean diameter of from about 1 nm to about 20 nm.

27. A composition for repairing or replacing bone in a subject in need thereof made by the method of claim 24.

28. A composition for repairing or replacing bone in a subject in need thereof made by the method of claim 25.

29. The composition of claim 1, wherein the repairing or replacing bone is in the oral cavity.

30. The method of claim 17, wherein the repairing or replacing bone is in the oral cavity.
